SEQUENCE LISTING (3 sequences)

SEQ ID NO: 1 - DNA, 59 bp, Artificial Sequence
  Synthetic promoter CP25
ctttggcagtttattcttgacatgtagtgagggggctggtataatcacatagtactgtt

SEQ ID NO: 2 - DNA, 12 bp, Artificial Sequence
  Ribosome-binding site
aggagatatacc

SEQ ID NO: 3 - DNA, 33 bp, Artificial Sequence
  Ribosome-binding site
aataattttgtttaactttaagaaggagatata

(19) United States
(12) Patent Application Publication
(10) Pub. No.: US 2014/0087435 A1
(43) Pub. Date: Mar. 27, 2014

(54) Novel Microbial Biocatalysts That Enables Use Of Cellodextrin As Biofuel

(71) Applicant: Georgia Tech Research Corporation, Atlanta, GA (US)

(72) Inventors: Rachel Ruizhen Chen, Marietta, GA (US); Hyun-Dong Shin, Johns Creek, GA (US); Ramanan Sekar, Atlanta, GA (US)

(73) Assignee: Georgia Tech Research Corporation, Atlanta, GA (US)

(21) Appl. No.: 13/966,173

(22) Filed: Aug. 13, 2013

Related U.S. Application Data
(60) Provisional application No. 61/682,506, filed on Aug. 13, 2012.

(51) Int. Cl. C12P 7/56

(52) U.S. Cl. CPC C12P 7/56
USPC 435/139; 435/252.33

(57) ABSTRACT

The present disclosure provides genetically engineered biocatalysts which enable intracellular assimilation of cellodextrin. The genetically engineered biocatalyst co-expresses a cellodextrin phosphorylase (CDP) gene and a cellobiose phosphorylase (CBP) gene. Further, the genetically engineered biocatalyst includes a first synthetic promoter used to express the cellodextrin phosphorylase (CDP) gene and a second synthetic promoter used to express the cellobiose phosphorylase (CBP) gene. Furthermore, the genetically engineered biocatalyst expresses one or more cellodextrin permeases. The intracellular assimilation includes hydrolysis and phosphorolysis mechanism. Further provided are methods of using the genetically engineered biocatalysts to generate various useful end-products including alcohol and lactic acid.

### CROSS-REFERENCE TO RELATED APPLICATIONS

This application claims the benefit under 35 U.S.C. §119(e) of U.S. Provisional Application No. 61/682,506, filed 13 Aug. 2012, which is incorporated by reference in its entirety.

### BACKGROUND

1. Field of the Technology

The present invention relates in general to genetically engineered biocatalysts. More particularly, it relates to biocatalysts which enable intracellular assimilation of cellodextrin.

2. Description of Related Art

Cellulosic materials are abundant renewable feedstocks which are potentially useful for production of biofuels and various other molecules. Their effective use could alleviate environmental concerns associated with petroleum feedstock and reduce the reliance of imported oil. The prevailing cellulosic technology requires cocktails of enzymes to completely de-polymerize cellulose to glucose before microbial fermentation. This requirement stems from the inability of microbial catalysts to use cellulose polymer directly. In fact, most microbial catalysts are unable to use even the much smaller partial hydrolysis products collectively known as cellodextrin (or glucose polymer with DP of 2 or higher). The concerted action of cellulases (endoglucanases and exoglucanases) yields a mixture of cellodextrin, whose further breakdown requires a β-glucosidase which releases a glucose molecule from cellobiose. As commercial cellulases are typically not adequate in β-glucosidase activities to produce sufficient glucose, its supplement is often found to be necessary (Breuil et al. 1992; Zhou et al. 2009). Overall, the demand of large amounts of enzymes is one of the most important obstacles in commercializing cellulosic technology (Lynd et al. 2005; Maki et al. 2009).

Researches aimed to reduce the amount required for β-glucosidase in hydrolysis, and generate a microbe capable of utilizing cellobiose, were reported for yeast (Wen et al. 2010) and other eukaryotes (Nakazawa et al. 2011). In most cases, a β-glucosidase was expressed extracellularly or displayed on cell surface to avoid the need to transport cellobiose into cells. Only limited success was achieved. While cells thus engineered were able to use cellobiose, the rate of product formation did not match what was from glucose (McBride et al. 2005; Tokuhiro et al. 2008). This may be due to the extra burden on cells for synthesis of glucosidase and limited extracellular expression or displayed enzyme. An alternative approach for direct assimilation of cellodextrin in yeast was reported recently, in which cellobiose intracellular assimilation was enabled by co-expression of a fungal major facilitator superfamily (MFS) transporter and β-glucosidase (Li et al. 2010). When used in simultaneous saccharification and fermentation (SSF), it increases consumption rates of glucose and cellobiose significantly, relative to a control without the transporter.

Researchers have also worked to engineer bacteria such as E. Coli (Wood et al. 1997; Zhou et al. 1999), Zymomonas mobilis, Klebsiella oxytoca (Zhou and Ingram 2001). To eliminate the need for extracellular β-glucosidase, the cellobiose operon from Klebsiella oxytoca has been cloned into E. Coli and expressed intracellularly, which encodes proteins in the PTS cellobiose uptake system and a phospho-β-glucosidase (catalyzing the hydrolysis of cellobiose-P into glucose and glucose-6-P). The resulting strain was able to ferment cellobiose into ethanol with about 90% yield without exogenous β-glucosidase supplement (Moniruzzaman et al. 1997). However, cellodextrin with degree of polymerization (DP) greater than two was not utilized due to the limitation of the PTS system.

Another strategy involved intracellular assimilation of cellobiose that allows use of phosphorolysis (Sadie et al, 2011; Sekar et al, 2012), an alternative cleavage mechanism to hydrolysis. This mechanism, commonly associated with cellulolytic bacteria, is explored in the present invention using a genetically engineered biocatalyst to construct potentially more energetic cells for cellodextrin metabolism.

### SUMMARY

The subject matter of this application may involve, in some cases, interrelated products, alternative solutions to a particular problem, and/or a plurality of different uses of a single system or article.

Disclosed herein are genetically engineered biocatalysts that enable intracellular assimilation of cellodextrin and the method thereof, wherein the genetically engineered biocatalyst express lacY or sde1395 or sde 2284 or a combination for uptake of cellobiose and additionally express a hydrolase (such as beta-glucosidase) or a cellobiose phosphorylase; and in the case for assimilation of cellodextrin larger than cellobiose, the biocatalyst express a permease (such as sde1395 and sde2284), and additionally co-express cellodextrin metabolizing enzymes such as a hydrolase (cellodextrinase) or a pair of phosphorylases, for example a cellodextrin phosphorylase and a cellobiose phosphorylase. Further, the genetically engineered biocatalyst includes a first synthetic promoter which is used to express the cellodextrin phosphorylase (CDP) gene. Furthermore, the genetically engineered biocatalyst includes a second synthetic promoter which is used to express the cellobiose phosphorylase (CBP) gene. Furthermore, the genetically engineered biocatalyst expresses one or more cellodextrin permeases. In one embodiment the genetically engineered biocatalyst is a bacterium. In an embodiment the bacterium is Escherichia coli (E. Coli). Further, the intracellular assimilation includes phosphorolysis and hydrolysis mechanism. Also, the intracellular assimilation evades carbon catabolite repression. The genetically engineered biocatalyst produces one or more fermentation end-products from the cellodextrin. The one or more fermentation end-products further include alcohol and lactic acid.

### BRIEF DESCRIPTION OF THE DRAWINGS

FIG. 1 provides a comparison of inhibitor tolerance in Hydrolysis and Phosphorolysis, in an embodiment of the present invention.

FIG. 2 provides tolerances of cellobiose-assimilating E. Coli strains against sodium acetate, in an embodiment of the present invention; (A) aerobically culture β-glucosidase (BGL); (B) aerobically culture cellobiose phosphorylase (CBP); (C) anaerobically culture BGL; and (D) anaerobically culture CBP.

FIG. 3 provides a sub-cloning process of CepB gene and Cep94A gene in phosphorolysis mechanism, in an embodiment of the present invention.

FIG. 4(a) provides a table depicting comparison of cell growth at initial level and cell growth of strain having phosphorylase gene and/or cellodextrin permease gene, in an embodiment of the present invention.

FIG. 4(b) provides a table depicting the comparison between empty vector and the strain expressing the transporter and phosphorylases, in an embodiment of the present invention.

FIG. 5 provides a table depicting comparison of ethanol productivity in mixture of glucose & xylose and mixture of cellobiose & xylose, in an embodiment of the present invention.

### DETAILED DESCRIPTION

The present invention may be understood more readily by reference to the following detailed description, the Examples/experiments included therein and to the Figures and their previous and following description.

Before the present compounds, compositions, articles, devices, and/or methods are disclosed and described, it is to be understood that the scope of the invention is not limited to specific synthetic methods, specific cellodextrin, or to particular gene, as such may, of course, vary. It is also to be understood that the terminology used herein is for the purpose of describing particular embodiments only and is not intended to be limiting. The following description, examples and experiments illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure.

Disclosed herein are genetically engineered biocatalysts that enable intracellular assimilation of cellodextrin and the method thereof, wherein the genetically engineered biocatalyst co-expresses cellodextrin phosphorylase (CDP) gene from C. thermocellum and cellobiose phosphorylase (CBP) gene from S. degradans. Further, the genetically engineered biocatalyst includes a first synthetic promoter which is used to express the cellodextrin phosphorylase (CDP) gene. Furthermore, the genetically engineered biocatalyst includes a second synthetic promoter which is used to express the cellobiose phosphorylase (CBP) gene.

In an embodiment of the present invention, the genetically engineered biocatalyst expresses one or more cellodextrin permeases. Further, in one embodiment, the genetically engineered biocatalyst is a bacterium. Furthermore, in an embodiment, the bacterium is Escherichia coli (E. Coli).

In one embodiment of the present invention, the intracellular assimilation includes phosphorolysis and hydrolysis mechanism. Also, the intracellular assimilation evades carbon catabolite repression. The genetically engineered biocatalyst produces one or more fermentation end-products from the cellodextrin. The one or more fermentation end-products further include alcohol and lactic acid.

In an embodiment of the present invention, biocatalysts metabolizing cellodextrin phosphorolytically are more productive and more robust as compared to hydrolysis mechanism. The phosphorolysis further allow much higher level of production of recombinant xylosidase and a green fluorescent protein.

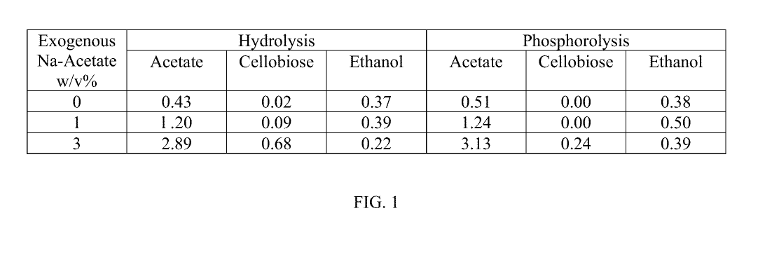

Referring now to FIG. 1, a table is shown which depicts results of experiments with two E. Coli constructs capable of cellobiose assimilation, in which LacY is the permease.

The experiments' results show the comparison between the hydrolysis and phosphorolysis mechanisms of cellobiose cleavage. The two E. Coli constructs are isogenic except that one is overexpressing beta-glucosidase (BglA) and the other overexpressing cellobiose phosphorylase (CepA). Since the phosphorolysis is more energy efficient, it produces more Adenosine Triphosphate (ATP) per glucose consumed in comparison to the hydrolysis mechanism. The cells expressing the CepA produce more recombinant enzymes than the cells expressing the BglA. When tested with a beta-xylosidase, it was found that the cells expressing the CepA produced approximately seven times more recombinant beta-xylosidase than the cells expressing the BglA, suggesting that the more energetic cells are more productive.

On comparison between the two types of cells on acetic acid tolerance, data showed that the cells expressing the CepA grow with a specific growth rate of 0.264 hr-1, under aerobic conditions with cellobiose as sole carbon source in the presence of 5% w/v sodium acetate, whereas the strain metabolizing cellobiose through the hydrolysis did not grow under otherwise the same conditions. At lower acetic acid concentration and anaerobic conditions, the cells undergoing the phosphorolysis of the cellobiose tolerated acetate better and produced more acetate as shown in FIG. 1. At 3% acetate, the cellobiose consumption was 32% for the hydrolysis and 76% for the phosphorolysis, with corresponding ethanol final titer of 22% and 39% for the hydrolysis and the phosphorolysis, respectively. Thus, the ethanol productivity gain by undergoing the phosphorolysis is by a factor of 2 for this specific case.

Results as depicted in FIG. 1 strongly support the notion that the phosphorolysis mechanism could be used to impart significant advantages in biofuel productions strains where synthesizing multiple enzymes and tolerance to inhibitors are crucial.

As analyzed above, one of the differences between the hydrolysis and the phosphorolysis of cellobiose is in the number of ATPs used in phosphorylation of cellobiose-derived glucose. The difference in ATP use was investigated to understand the impact on cells' acetate tolerance.

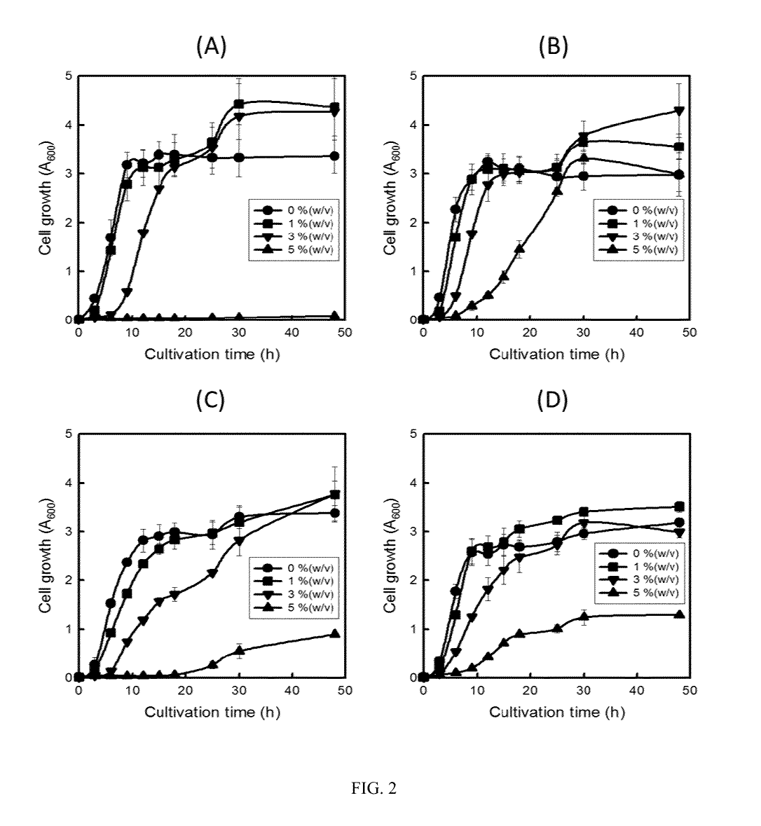

Furthermore, referring to FIG. 2, to compare tolerances of engineered strains against acetate, which is a major inhibitor derived from biomass, β-glucosidase (BGL) and cellobiose phosphorylase (CBP) strains were aerobically cultivated in Luria broth (LB) medium having 1% (w/v) cellobiose and different concentration of Na-acetate ranging from 0 to 5% (w/v) at 370C and 250 rpm for 2 days. Cell growth was monitored by sampling from the cultures and measuring the OD600 values at periodic time intervals. Both strains, the CBP and the BGL strains were also aerobically cultivated in LB medium having 1%(w/v) cellobiose and different concentration of n-butanol ranging from 0 to 1.0%(w/v) at 370C and 250 rpm for 60hrs. Cell growth was spectrophotometrically determined at 600 nm. Cells assimilating cellobiose phosphorolytically show better tolerance for common inhibitors such as acetate and butanol.

A set of aerobic experiments were carried out under the different conditions of the LB Medium. The medium was supplemented with different concentrations of sodium acetate.

Compared to the control without addition of sodium acetate, for the both strains, cells grown in the presence of the acetate inhibitor at concentrations of 1% and 3% (w/v) exhibited two growth phases, first growth phase (0-24 hrs) was on cellobiose, and the second phase was due to assimilation of acetate (FIG. 2A and 2B). Careful examination of the growth profile showed that at both 0% and 1% acetate concentration, the BGL cells reached slightly higher final cell density than that of the CBP cells. At 3% sodium acetate concentration, however, the CBP cells had comparable final cell density with the BGL cells. There was also a smaller lag time for the CBP cells than the BGL cells. More difference was observed when the cells were cultivated with 5% sodium acetate. The BGL cells did not show any sign of growth during the 48 hours of cultivation (FIG. 2A). In contrast, the CBP cells were able to grow after a lag time of about 6 hours with growth rate lower than the cases with lower acetate concentration (FIG. 2B). The final cell density, just above Optical Density (OD) 3, was only slightly lower than what achieved at lower acetate concentration (FIG. 2B). The dramatic difference between the two strains at 5% sodium acetate was surprising as it was expected that the impact of different use of ATP between the two strains on overall cellular energetic should be minimal especially under aerobic condition.

The experiment was repeated several times and the same results were obtained. The lack of cell growth for the BGL cells at 5% sodium acetate concentration was observed in reference to growth profiles obtained under anaerobic conditions (FIG. 2C) as at the same concentration of acetate, the BGL cells were able to grow after 18 hours of lag time.

Compared to aerobic cells, the growth profiles for the both strains under anaerobic conditions were different in that only one growth phase was evident. This was supported by the measurement of acetate, which showed minimal consumption under anaerobic conditions and some consumption under aerobic conditions. The difference between the two strains at 3% inhibitor concentration was subtle. The CBP cells had about 2 hours shorter lag time than the BGL cells, as shown in FIG. 2D. However, the BGL cells reached slightly higher final cell density. More difference between the two strains was observed at 5% sodium acetate concentration; the CBP cells had a lag time of 6 hours which is much shorter than 18 hours for the BGL cells. Additionally, the CBP cells reached a higher cell density, 1.2 versus 0.8 for the BGL cells. Surprisingly, these results clearly showed that the CBP cells tolerate acetate better, despite the fact that it may not be attributed solely to energetics.

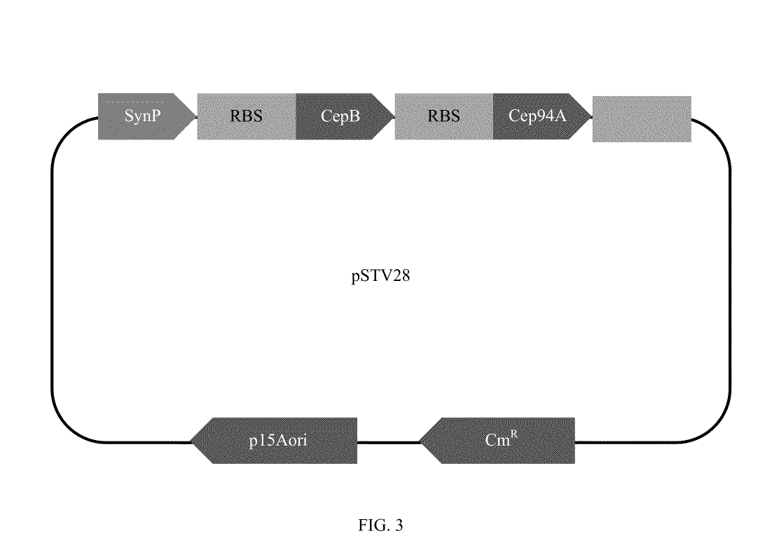

In another embodiment of the present invention, referring to FIG. 3, a process flow is shown for co-expression of cellodextrin phoshorylase gene from Clostridium thermocellum and cellobiose phosphorylase gene from Sacchrophagus degradans to enable E. Coli to use cellodextrin. The engineered strain was able to grow with the cellodextrin as sole carbon source.

In preferred embodiment of the present invention, to utilize the cellodextrins as the carbon source through phosphorolysis , CepB gene encoding cellodextrin phosphorylase ( CDP, GenBank: AB006822.1) of C. thermocellum and Cep94A gene (cellobiose phosphorylase, CBP, Gene Bank: NC_{—}007912) from S. degradans are subcloned behind a Synthetic Promoter (SynP) in pSTV28 vector as shown in FIG. 3. Synthetic promoter, CP25 (5′-CTTTGGCAGTTTATTCTTGACATGTAGTGAGGGGGCTGGTATAATCACATAGTAC TGTT-3′, which was developed by Jansen and Hammer (1998. Appl. Environ. Microbiol. 1998. 64(1): S2-87), was used for expression of both the CepB and the Cep94A genes. The sequence of ribosome-binding site (RBS) for the CepB gene and the Cep94A are 5′-AGGAGATATACC-3′ (RBS of ET2Ob(+), Novagen) and 5′-AATAATTTTGTTTAACTTTAAGAAGGAGATATA-3′ (Dortay et al. PLoS ONE 6 (4), El 8900 (201 1)).

In an embodiment of the present invention, recombinant plasmid was transformed into E. Coli SZ63 producing (D)-lactic acid and the successful expression of two phosphorylase genes were confirmed by enzyme activity assay. The plasmid was also co-transformed to E. Coli SZ63 with one of two cloned cellodextrin permeases, Sde-2284 and Sde-139.5 genes in pBBR 122.

In one embodiment, the engineered strains were firstly cultivated in LB medium with 0.2 mM IPTG to co-express the two phosphorylase gene and cellodextrin (CD) permease gene and then transferred to M9-CD medium to ferment CDs to lactic acid

In another embodiment, all engineered SZ63 strain having phosphorylase gene and/or CD permease gene were aerobically cultivated in M9˜medium with 1%(w/v) Celloteraose (G4) at 37° C. for 48h. For this experiment, HPLC glass vials having 0.21771 of the M9 culture medium were used. After 2 days, cell growth was determined by a microplate reader.

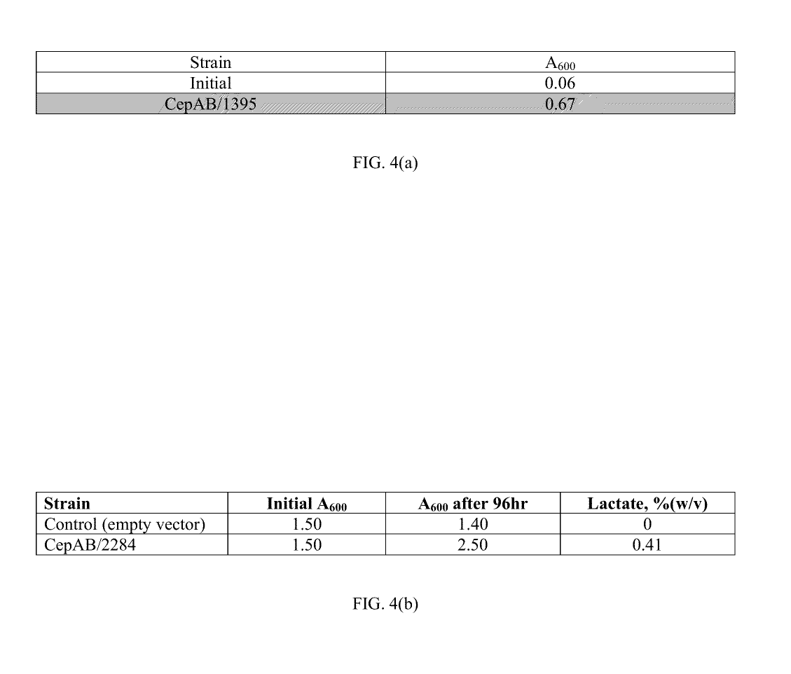

As shown in table depicted in FIG. 4(a), co-expression of transporter and phosphorylases enabled significant cell growth. The OD was increased from initial 0.06 to 0.67.

In yet another embodiment of the present invention, to produce lactic acid from cellodextrin mixture by the engineered E. Coli SZ63 co-expressing the CepB gene encoding cellodextrin phosphorylase (CDP) of Clostridium thermocellum and the Cep94A gene (cellobiose phosphorylase, CBP) from S. degradans, this was firstly cultivated at 37° C. in LB medium with 0.2 mM IPTG for overnight to prepare the cells expressing both enzymes. The cells were inoculated to the M9 minimal medium having 1%(w/v) cellodextrins and 0.2 mM IPTG and then incubated anaerobically for 96 hr as shown in table depicted in FIG. 4(b).

Referring again to FIG. 4(b), a cell growth comparison of the control with empty vector and the strain expressing the transporter and both phosphorylases is provided. As shown in the table of FIG. 4(b), the strain expressing the transporter and both phosphrylases were able to grow under anaerobic condition and additionally produced lactic acid, surprisingly demonstrating the utility of the biocatalysts engineered to convert the cellodextrin to useful products through phosphorolytic mechanism.

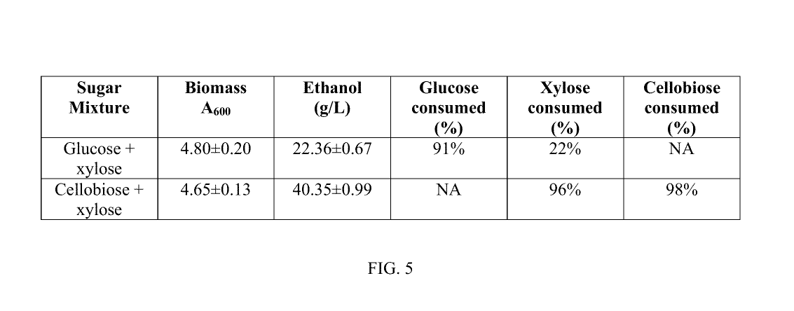

In another embodiment, referring to FIG. 5, intracellular cellobiose utilization using genetically engineered biocatalyst evades catabolite repression, allowing simultaneous fermentation of xylose and cellobiose, resulting in complete sugar utilization. In an exemplary embodiment, the intracellular assimilation is used to evade carbon catabolite repression, a mechanism that prevents simultaneous utilization of glucose and xylose, the two most prevalent sugars in plant biomass. The intracellular uptake of the cellodextrin and its subsequent metabolism mask the presence of the glucose, thus allow the cellodextrin and the xylose to be simultaneously utilized in a fermentation process thereby significantly reduce the fermentation time and enhance productivity. This was observed in a mixed fermentation with (5% glucose +5% xylose) or (5% cellobiose +5% xylose), with an E. Coli biocatalyst engineered to assimilate cellobiose through hydrolysis route (expressing BglA from S. degradans). The simultaneous consumption of the cellobiose and the xylose was observed with the mix of the cellobiose and the xylose, in contrast with the sequential use of monosaccharides in the case of the glucose and the xylose. As a result, much more ethanol was produced for the cellobiose/the xylose than for glucoselxylose during the 140 hours of fermentation. As shown in table depicted in FIG. 5, only 20% of the xylose consumption was achieved in the mixed monosaccharide fermentation, compared to a near complete xylose consumption in the case of the cellobiose/the xylose, a five-fold difference. The productivity gain from simultaneous consumption of the xylose and the cellobiose as opposed to sequential use of the glucose and the xylose is about two-fold.

While preferred embodiments of the present invention have been shown and described herein, it will be obvious to those skilled in the art that such embodiments are provided by way of example only. Numerous variations, changes, and substitutions will now occur to those skilled in the art without departing from the invention described herein. It should be understood that various alternatives to the embodiments of the invention described herein can be employed in practicing the described subject matter. It is intended that the following claims define the scope of the invention and that methods and structures within the scope of these claims and their equivalents be covered thereby.

What is claimed is:

1. A genetically engineered biocatalyst that enables an intracellular assimilation of cellodextrin, wherein the genetically engineered biocatalyst comprises:
a cellodextrin phosphorylase (CDP) gene expression of a corresponding gene from C. thermocellum; and
a cellobiose phosphorylase (CBP) gene expression of a corresponding gene from S. degradans.

2. The genetically engineered biocatalyst of claim 1, wherein the intracellular assimilation comprises a phosphorolysis mechanism, the phosphorolysis mechanism being inherent to a CepB gene encoded cellodextrin phosphorylase of a C. thermocellum and a Cep94A gene encoded cellobiose phosphorylase from a S. degradans.

3. The genetically engineered biocatalyst of claim 1, wherein the intracellular assimilation comprises a hydrolysis mechanism, the hydrolysis mechanism being inherent to a beta-glucosidases.

4. The genetically engineered biocatalyst of claim 1, wherein the intracellular assimilation evades a carbon catabolite repression.

5. The genetically engineered biocatalyst of claim 1, wherein the genetically engineered biocatalyst produces one or more fermentation end-products from the cellodextrin.

6. The genetically engineered biocatalyst of claim 5, wherein the one or more fermentation end-products comprises an alcohol.

7. The genetically engineered biocatalyst of claim 5, wherein the one or more fermentation end-products comprises a lactic acid.

8. The genetically engineered biocatalyst of claim 1, wherein a first synthetic promoter is used for the cellodextrin phosphorylase (CDP) gene expression.

9. The genetically engineered biocatalyst of claim 8, wherein the first synthetic promoter is CP25 (5′-CTTTGGCAGTTTATTCTTGACATGTAGTGAGGGGGCTGGTATAATCACATAGTAC TGTT-3′).

10. The genetically engineered biocatalyst of claim 1, wherein a second synthetic promoter is used for the cellobiose phosphorylase (CBP) gene expression.

11. The genetically engineered biocatalyst of claim 10, wherein the second synthetic promoter is CP25 (5′-CTTTGGCAGTTTATTCTTGACATGTAGTGAGGGGGCTGGTATAATCACATAGTAC TGTT-3′.

12. The genetically engineered biocatalyst of claim 1, wherein the genetically engineered biocatalyst expresses one or more cellodextrin permeases.

13. The genetically engineered biocatalyst of claim 12, wherein the one or more cellodextrin permeases comprises a Sde-2284 and a Sde-1395 genes in pBBR122.

14. The genetically engineered biocatalyst of claim 1, wherein the genetically engineered biocatalyst is a bacterium.

15. The genetically engineered biocatalyst of claim 12, wherein the bacterium is an Escherichia coli (E. Coli).

16. A genetically engineered biocatalyst, wherein the biocatalyst enables an intracellular assimilation of cellodextrin, the intracellular assimilation comprising of a phosphorolysis mechanism and a hydrolysis mechanism, such that the intracellular assimilation evades a carbon catabolite repression.

17. A method of using a genetically engineered biocatalyst for an intracellular assimilation of cellodextrin, wherein the genetically engineered biocatalyst co-expresses cellodextrin phosphorylase (CDP) gene from C. thermocellum and cellobiose phosphorylase (CBP) gene from S. degradans.
